(19) United States
(12) Patent Application Publication
(10) Pub. No.: US 2005/0231686 A1
(43) Pub. Date: Oct. 20, 2005

(54) Device for detecting measurands in an eye

(75) Inventor: Christian Rathjen, Bremen (DE)

Correspondence Address: OLIFF & BERRIDGE, PLC, P.O. BOX 19928, ALEXANDRIA, VA, 22320, US

(73) Assignee: SIS AG, SURGICAL INSTRUMENT SYSTEMS, Allmendstrasse11 (CH)

(21) Appl. No.: 10/513,776

(22) PCT Filed: Jun. 26, 2003
(86) PCT No.: PCT/CH03/00427

§ 371 Date: Nov. 9, 2004

(30) Foreign Application Priority Data
Jun. 27, 2002 (EP) 02 405 542.8

(51) Int. Cl. A61B 3/10

(52) U.S. Cl. 351/205

(57) ABSTRACT

Proposed is a device (1) for detecting measurement data of an eye, in particular a human eye. The device comprises a support (11) and a sensor (12) fixed to the support (11) for detecting measurement data on the eye. The support (11) is provided with fastening means (11a, 11b) for fastening the device (1) to at least one finger (2) of a user. The support (11) is designed such that the sensor (12) is positionable in the region of the fingerpad (22) of a finger (2), the sensitive region of the sensor (12) being remote from the fingerpad (22). The length of the part of the support (11) situated in the region of the fingerpad (22) is limited to the length of the distal phalanx (21), situated there, of the finger (2). The sensor (12) is fixed to the support (11) in such a way that in the state of the support (11) fastened to the finger (2) a pressing force arising during an application of the sensor (12) to the eye is transmittable to the finger (2).

### TECHNICAL FIELD

The present invention relates to a device for detecting measurement data of an eye. The invention relates in particular to a device for detecting measurement data of a human eye, which device comprises a support and a sensor fixed to the support for detecting the measurement data on the eye.

### PRIOR ART

In the field of ophthalmology, many different devices are known for detecting measurement data on a human eye. With the advances made in the field of sensor technology, particularly in micro-electronic and micro-electromechanical sensor technology, it became possible to detect measurement data on the human eye with the aid of sensors rather than by means of mechanical measuring devices. Ophthalmic measuring devices with miniaturized pressure sensors or force sensors have been developed and are increasingly used, particularly for determining what is called the intraocular pressure (IOP). Even with the use of these miniaturized sensors, however, the known ophthalmic measuring devices are generally too large and unwieldy and, in particular, unsuitable for self-application by the user.

A device for measuring intraocular pressure is described in U.S. Pat. No. 3,272,001. The device according to U.S. Pat. No. 3,272,001 comprises a measurement probe and an air pump and a pressure display, which are connected to the measurement probe via air ducts. The measurement probe according to U.S. Pat. No. 3,272,001 can be fixed on a user's finger by means of a ring or a clip. The measurement probe according to U.S. Pat. No. 3,272,001 comprises a mechanical plunger which can be applied to the eye and which transmits the intraocular pressure to the pressure display via the air ducts.

### DISCLOSURE OF THE INVENTION

It is an object of the present invention to propose a novel device for detecting measurement data on an eye, in particular a human eye, which device does not have the disadvantages of the prior art and is particularly easy to handle and is suitable for self-application by the user.

According to the present invention, these aims are achieved in particular by the elements of the independent claims. Further advantageous embodiments are also set out in the dependent claims and the description.

The abovementioned aims are achieved by the present invention in particular by virtue of the fact that in the device for detecting measurement data on an eye, in particular a human eye, which device comprises a support and a sensor fixed to the support for detecting the measurement data on the eye, the support is provided with fastening means for fastening the measurement device to at least one finger of a user. The fastening of the sensor on a finger of the user permits positioning and application of the sensor with the aid of the high degree of manual motor ability of a human, without the assistance of complex drives, for example as in the known automated Goldmann tonometers. Thus, the possible safety risks of automated positioning and measurement methods are also eliminated. The compact and light design of the measurement device for fastening to a user's finger also reduces the risk of injury. During the application of the sensor by means of a finger of the user, protective reflexes can also be optimally exploited: the withdrawal of the hand or of the finger is quicker than the withdrawal of the head or upper body. The fastening of the measurement device on a finger of the user also permits, in particular, self-application.

The support is preferably designed in such a way that the sensor is positionable in the region of the fingerpad of a finger, the sensitive region of the sensor being remote from the fingerpad. By positioning the sensor in the region of the fingerpad, which includes the finger tip and the finger pulp, the sensor comes to lie at the finger end, where a human has, both in tactile and also motor terms, the greatest degree of sensitivity for the application and the greatest degree of dexterity. Particularly in the case of self-application, this permits precise and sensitive positioning of the sensor on the eye. By positioning the sensor in the region of the fingerpad, an extremely stable reference can be generated by placing the ball of the thumb of the relevant hand on the cheek or the chin and/or by placing the middle phalanx of the relevant finger on the cheek bone, this reference greatly minimizing the shaking and wobbling of the sensor and the associated risk of injury and permitting simple and precise application of the sensor on the eye without additional positioning means.

The sensor is preferably fixed to the support in such a way that in the state of the support fastened to the finger a pressing force arising during an application of the sensor to the eye is transmittable to the finger. Thus, the user perceives the pressing force caused by application of the sensor to the eye directly through the sense of touch, and the user is able to use acquired human motor skills for the application and can intuitively sense the force needed for the application and the necessary movements and their actual magnitude. It is not necessary to learn other indirect parameters, for example the path of displacement of a spring-mounted contact body.

In one embodiment variant, the measurement device comprises structural elements which are disposed behind the sensor and remote from the sensitive region of the sensor, and which are perceivable on the finger by the user in the state of the support fastened to the finger. By means of such structural elements, the user can tell the exact position of the sensor on his finger and the pressing force arising during application of the sensor to the eye.

In one embodiment variant, the measurement device comprises an application ring which encircles the sensor and which abuts the eye during the detection of the measurement data. Such an application ring serves as application aid, on the one hand, by preventing sharp edges of the sensor from contacting the eye and damaging the eye surface upon application of the measurement device, and, on the other hand, by serving as a centering aid.

Preferably, in the state of the support fastened to the finger, the length of the part of the support situated in the region of the fingerpad is limited to the length of the distal phalanx, situated there, of the finger. By limiting the extent of the support to the length of the distal phalanx of the finger, the mobility of the distal phalanx on which the sensor is fixed is not restricted and the highest degree of mobility of the relevant finger is afforded.

In one embodiment variant, the sensor is movably mounted on the support. Mounting the sensor movably on the support has the effect that, upon application with contact on the eye, the sensor rests better on the eye and, in this way, a slightly skew application can be corrected.

In one embodiment variant, the measurement device comprises means for attaching a disposable protective membrane for covering the sensor. The use of a protective membrane over the sensor reduces the risk of injury to the eye by the sensor. The easy exchangeability of protective membranes, made possible by these attachment means, reduces the risk of soiling of the sensitive region of the sensor and increases hygiene in the use of the measurement device.

In one embodiment variant, the measurement device comprises an interface module fixed to the support for data communication, with contact or contactless, with an evaluation unit external to the measurement device. Such an interface module permits storage, processing and evaluation of detected measurement data in a unit external to the measurement device, so that the size and weight of the measurement device can be reduced.

In one embodiment variant, the measurement device comprises processing means, a data store, a display and/or an energy source, which are arranged on the support. This permits independent use of the measurement device without necessary connection to additional external units, which is preferable especially when using the measurement device for self-application.

In one embodiment variant, the support is designed as a bow comprising a curved area which rests on the finger tip in the state fastened to the finger. This embodiment is particularly flexible because the support can be adapted to different shapes and sizes of fingerpads.

In one embodiment variant, the support is designed as a thimble. This embodiment ensures a particularly good hold of the measurement device on the finger of the user.

In various embodiment variants, the fastening means comprise a fastening clamp, an adhesive closure, an elastic band, a ring or a spreader ring. Different preferences on the part of the users can be taken into account by providing different embodiments of the fastening means. With adjustable fastening means such as spreader rings, elastic bands or adhesive closures, the measurement device can also be fastened on different fingers and/or on several fingers.

In different embodiment variants, the sensor can also comprise a pressure sensor array, further a force sensor, a contact sensor, a distance sensor, a chemosensor, a surface sensor, a temperature sensor and/or a micro-optical emitter-receiver module.

In one embodiment variant, the sensor comprises a light source as an optical application aid. In this way, in addition to the tactile feedback, the user can also be provided with an optical signal as an application aid, this signal indicating to the user the distance of the sensor from the eye and/or the contact of the sensor with the eye.

In one embodiment variant, the measurement device comprises an electro-acoustical converter as an acoustical application aid. In this way, in addition to the tactile feedback, the user can also be provided with an acoustic signal as an application aid, this signal indicating to the user that the sensor is approaching the eye.

### BRIEF DESCRIPTION OF THE DRAWINGS

An embodiment of the present invention is described below with reference to an example. The example of the embodiment is illustrated by the following figures attached:

FIG. 1 shows a plan view of a device for detecting measurement data on an eye, which device is fixed on the distal phalanx of a finger.

FIG. 2 shows a side view of a device for detecting measurement data on an eye, which device is fixed on the distal phalanx of a finger.

FIG. 3 shows a perspective view of a device for detecting measurement data on an eye, which device comprises a support designed as a thimble.

FIG. 3a shows a side view of a device for detecting measurement data on an eye, which device comprises a support designed as a thimble on which a sensor is fixed.

FIG. 3b shows a side view of a device for detecting measurement data on an eye, which device comprises a support designed as a thimble on which a sensor is fixed in an alternative arrangement.

FIG. 3c shows a side view of a device for detecting measurement data on an eye, which device comprises a support designed as a thimble at whose vertex a sensor is fixed.

FIG. 4 shows a perspective view of a device for detecting measurement data on an eye, which device comprises a support designed as a bow with a curved area which rests on the finger tip in the state fastened on the finger.

FIG. 4a shows a side view of a device for detecting measurement data on an eye, which device comprises a support designed as a bow with a curved area which rests on the finger tip in the state fastened on the finger.

FIG. 5a shows a side view of a device for detecting measurement data on an eye, which device comprises a support designed as a thimble on which a protective membrane is fixed which covers a sensor mounted on the support.

FIG. 5b shows a cross section through a support with a sensor mounted thereon, which is encircled by an application ring and is covered by a protective membrane fastened on the application ring.

FIG. 6 shows a plan view of the sensitive region of a sensor which is encircled by an application ring and comprises a centrally disposed pressure sensor element and several contact sensor segments.

FIG. 7 shows a block diagram which schematically illustrates an electronics module.

### EMBODIMENTS OF THE INVENTION

In the figures, parts corresponding to one another are designated by the same reference labels.

Reference label 1 designates a device for detecting measurement data on an eye, in particular a human eye. The measurement device 1 comprises a support 11, a sensor 12 fixed thereon, and an electronics module 17 connected to the sensor 12.

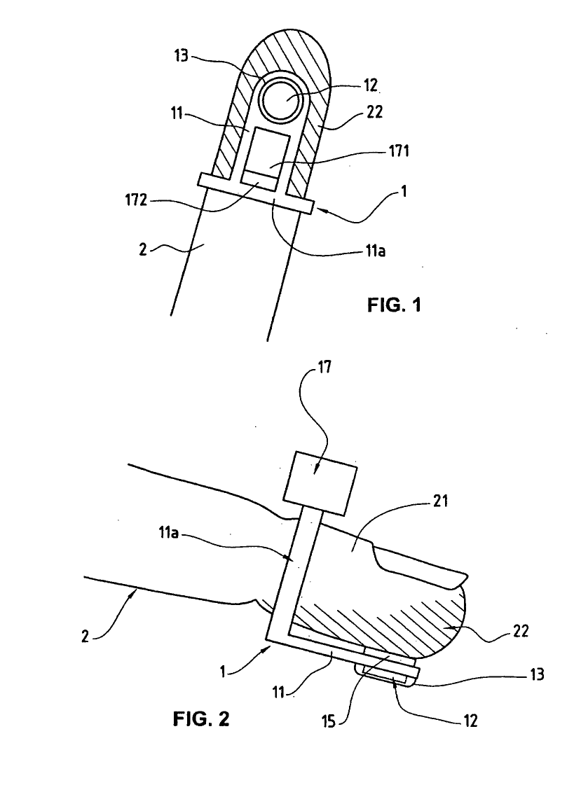

As is shown in FIGS. 1 and 2, the measurement device 1 comprises a ring 11a fastened to the support 11 for the purpose of fastening the measurement device 1 on a finger 2 of a user, for example on the index finger. As is shown in FIGS. 1 and 2, the sensor 12 is arranged at the free rounded end of the tongue-shaped support 11 and, when the measurement device 1 is in the state fastened to the finger 2, comes to lie in the region of the fingerpad 22 of the finger 2. As is indicated in FIG. 2, the region of the fingerpad 22 comprises the finger tip and the finger pulp of the finger 2. The sensor 12 is fixed on that side of the support 11 remote from the finger 2. On the side of the support 11 facing the finger 2, structural elements 15 are disposed behind the sensor 12 on the support 11. Via the structural elements 15, the user perceives, through his sense of touch, the position of the sensor 12 and the pressing force arising upon application of the sensor 12 to the eye. The structural elements 15 are perceptible elevations of low height, for example 0.5 millimeter. The sensitive region of the sensor 12 is remote from the support 11, so that it can be applied to an eye by the user using his finger 2, by pressing it onto the eye.

As is shown in FIG. 2, the support 11 is so dimensioned that it does not extend beyond the length of the distal phalanx 21 of the finger 2, so that the mobility of the distal phalanx 21 is not restricted.

The sensor 12 is preferably mounted movably on the support 11, but the pressing force arising upon application of the sensor 12 to the eye is still transmittable from the sensor 12 to the fingerpad 22 via the support 11 and the structural elements 15. Various fastening means suitable for this purpose are known to the skilled person. The sensor 12 can, for example, be arranged on the support 11 via a universal joint, e.g. as a thin injection-molded part of plastic (elasto-kinematic hinge). The sensor 12 can also be fixed on the support 11 via a suspension in a membrane 19 fastened to the support 11, e.g. as is done in rubber keys of remote controls. The sensor 12 can also be fastened to the support 11 via a tension spring and mounted in a concave or convex socket. A further possibility is to arrange the sensor 12 on the support 11 on a flexible cushion, e.g. of foam, an air cushion or silicone cushion, fastened to the support 11.

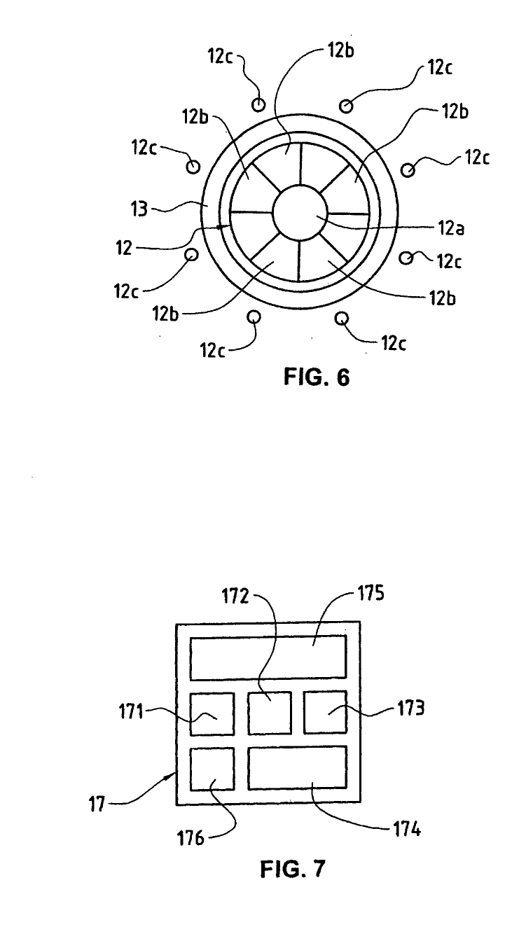

As is illustrated in FIG. 6, the sensor 12 can comprise several sensor elements 12a, 12b. The sensitive region of the sensor 12 shown in FIG. 6 comprises a centrally disposed pressure sensor element 12a and several contact sensor segments 12b, for example leakage capacitors, which are arranged concentrically about the pressure sensor element 12a. The pressure sensor element 12a in turn consists of several pressure sensors arranged in an array. The pressure sensor element 12a serves for determining the intraocular pressure. The contact sensor elements 12b deliver contact signals which are used for application assistance by signaling a correct bearing of the sensor 12. As is indicated diagrammatically in FIG. 6, the individual contact sensor segments 12b are each geometrically assigned to light sources 12c, for example LEDs (light-emitting diodes), in particular multi-color LEDs. The light sources 12c are each controlled dependently on the contact signal of the respectively associated contact sensor segment 12b. The light sources 12c are arranged in such a way that the light emitted from the light sources 12c can be seen directly by the user from the light source 12c and/or as a reflection on the eye surface. In combination with preferably concentrically arranged distance sensors, the light sources 12c can additionally be used as an application aid for maintaining a certain distance from the eye. An individual central light source could also be used. A corresponding application aid can also be achieved by graphic representation of the contact signals, or distance signals, on a display.

In addition to pressure sensor arrays and contact sensors, the sensor 12 can also be equipped with force sensors, distance sensors, chemosensors, surface sensors, temperature sensors and/or micro-optical emitter-receiver modules, so that, in addition to determination of the intraocular pressure, it is also possible to determine pulse, body temperature of the ocular fundus, blood circulation in the eye, and various biological markers of the lachrymal fluid, retina, anterior chamber fluid, cornea or Schlemm's canal, and, in addition to determination of the eye contact as an application aid, it is also possible to determine the distance from the eye and the contact surface as an application aid. At this point it should be noted that the application of the sensor 12 for determining the intraocular pressure involves contact of the sensor 12 with the eye (in particular with the cornea, the lid or the sclera), whereas, upon determination of other physiological measurement data, for example determination of pulse or blood circulation, the sensor 12 is applied while maintaining a certain distance from the eye.

Although this has not been shown, it should be noted here that the measurement device 1 can additionally be provided with actuator elements which are disposed behind the sensor 12, remote from the sensitive region of the sensor 12, in such a way that they are perceivable on the finger 2 by the user when the measurement device 1 is in the state fastened to the finger 2. The measurement device 1 can additionally be provided with a driver module which controls the actuator elements as a function of the measurement signals detected by the sensor 12. In this way, an active force feedback can be achieved between finger 2 and sensor 12 for increasing the sensed pressing force or for active alignment of the sensor 12.

As is shown schematically in FIG. 7, the electronics module 17 comprises processing means 171, a data store 172, an interface module 173, an energy source 174 (e.g. a battery), a display 175 and an electro-acoustic converter 176. The electronics module 17 can also be made simpler. In order not to impair the mobility of the distal phalanx, the electronics module 17 is arranged on the side remote from the fingerpad 22 and, as is shown in FIG. 2, for example fastened to the ring 11a. The electronics module 17 can be connected removably to the measurement device 1 so that it can be electrically coupled to different types of sensors 12, in which case different sensor types can be identified by identification codes which are detectable by the electronics module 17 via the removable connection to the measurement device 1. As is shown in FIG. 1, certain parts of the electronics module 17 can also be formed in the support 11: the processing means 171 and the data store 172 can be integrated into the support 11, for example in CMOS (complementary metal oxide semi-conductor) technology. In one embodiment variant, the processing means 171 and data store 172 and also the sensor 12 are integrated in a common CMOS chip.

The processing means 171 comprise analog-digital converters for converting the analog measurement signals received by the sensor 12 to digital measurement data, and also a processor or logic module. If the measurement signals of the sensor 12 are generated optically, for example by interferometry, the measurement signals can be transmitted to the electronics module 17 via light guides. In this case, the measurement device 1 additionally comprises electro-optical converters. In addition to storing the detected measurement data, the data store 172 may also be used to store programmed software modules for controlling the processor. The processing means 171 process and scale the measurement signals received by the sensor 12 and deliver them to the display 175, the electro-acoustical converter 176 and/or the interface module 173. The interface module 173 comprises contacts for coupling to a processing unit external to the measurement device 1, or an emitter module for contactless data transmission to this external processing unit. The display 175 is used to depict measurement data and/or optical application aids. The electro-acoustical converter 176 serves for audible reproduction of signals for the application aid, for example different tones and/or volumes as a function of the distance of the sensor 12 from the eye. The energy source 174 comprises a battery, a photovoltaic solar cell, or an attachment for a supply unit external to the measurement device 1.

In one embodiment variant, the display 175 can be arranged in a housing separate from the support 11 and can be provided with fastening means for fastening it to an arm of the user, so that it can be worn like a wristwatch by the user. For data exchange, the display 175 arranged in this way is connected to the interface module 173 either contactlessly or with contact. The same housing in which the display 175 thus arranged is contained can also accommodate the processing means 171, the data store 172 and/or the energy source 174.

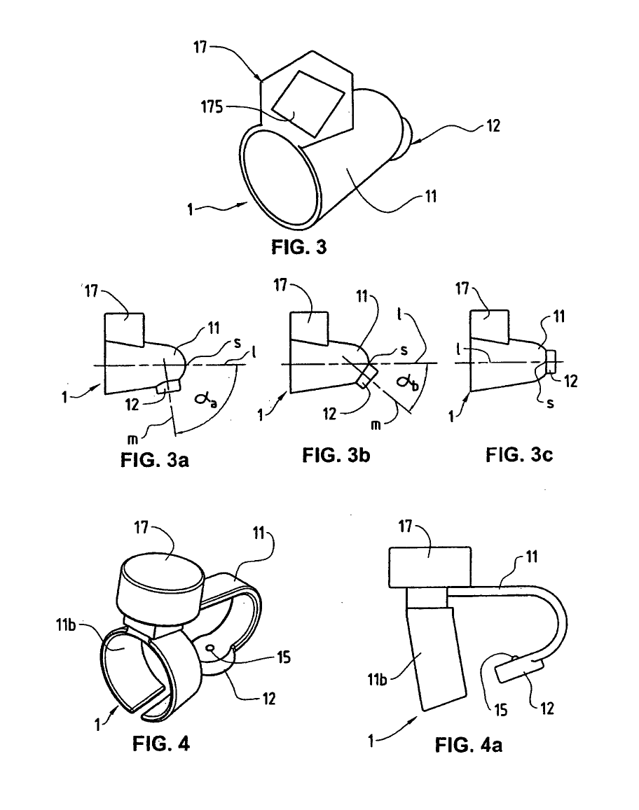

FIG. 3 shows a measurement device 1 for detecting measurement data on an eye, the support 11 being designed as a thimble. The support 11 designed as a thimble can be pushed over the distal phalanx 21 of the finger 2 and can be of a rigid design, for example of plastic, or of a flexible design, for example of rubber. FIG. 3 also shows an embodiment variant in which the display 175 is fixed on an angled surface of the electronics module 17 in such a way that, during application of the sensor 12, it can be easily seen by a second person. However, by storing measurement data, the measurement data recorded can also be shown to the user on the display 175 after self-application.

FIGS. 3a, 3b and 3c show different embodiment variants of the measurement device 1 in which the sensor 12 is in each case arranged in a different way on the support 11 designed as a thimble. In the embodiment variant according to FIGS. 3a and 3b, the center axis m of the sensor 12 is inclined by an angle αₐ and α_{b}, respectively, from the longitudinal axis I of the support 11, the angle αₐ being approximately 80° and the angle α_{b} approximately 40°. In the embodiment variant according to FIG. 3c, the sensor 12 is arranged axially on the vertex S of the support 11 designed as a thimble. The support 11 designed as a thimble can in each case be turned on the finger in such a way that, in the state of the measurement device 1 fastened to the finger 2, the sensor 12 comes to lie in the region of the fingerpad 22 of the finger 2. However, the different arrangements of the sensor 12 according to FIGS. 3a, 3b and 3c involve different applications by the user. The arrangement according to FIG. 3a permits a more stable and more comfortable application, centrally on the cornea, than do the other two arrangements according to FIGS. 3b and 3c, since, on the one hand, the finger 2 can be used in an uncurved position and, on the other hand, it permits better support of the ball of the thumb and of the finger 2 on the cheek and on the cheek bone, respectively, of the person to be examined. The arrangement according to FIG. 3b is, for example, better suited if application is on the sclera between nose and cornea or if the measurement device 1 is held without contact directly in front of the cornea. Different measurement devices 1 with different arrangements of the sensor 12 according to FIGS. 3a, 3b and 3c can be used by different users with different user preferences.

The arrangements of the sensors 12 shown in FIGS. 3a and 3b can also be combined to give a design of the device 1 with two different sensors 12, in which case, for example, a first sensor 12 for measuring the intraocular pressure lies on the eye and a second sensor 12 for measuring the blood oxygen value is situated without contact in front of the eye.

FIGS. 4 and 4a show a device 1 for detecting measurement data on an eye, which device comprises a support 11 designed as a bow having a curved area which rests on the finger tip when in the state fastened to the finger. The support 11 designed as a bow is connected at one end to a spreader ring 11b which is pushed over the distal phalanx 21 of the finger 2. At the free end of the support 11 designed as a bow, the sensor 12 is arranged in such a way that, when the measurement device 1 is in the state fastened to the finger 2, it comes to lie in the region of the fingerpad 22 of the finger 2. The above-described structural elements 15 are arranged behind the rear face of the sensor 12, remote from the sensitive region of the sensor. The spreader ring 11b is preferably connected removably to the support 11 so that spreader rings 11b of different size can be connected to the support 11 to permit adaptation to different finger sizes.

Compared to the embodiment variant of the measurement device 1 shown in FIGS. 1 and 2, the embodiment variants of the measurement device 1 shown in FIGS. 3, 3a, 3b, 3c, 4 and 4a have a more stable fastening on the finger 2. In contrast to the first embodiment variant, however, those areas of the support 11 of the latter embodiment variant lying in the region of the finger tip may represent an obstacle upon application of the sensor 12 to the eye.

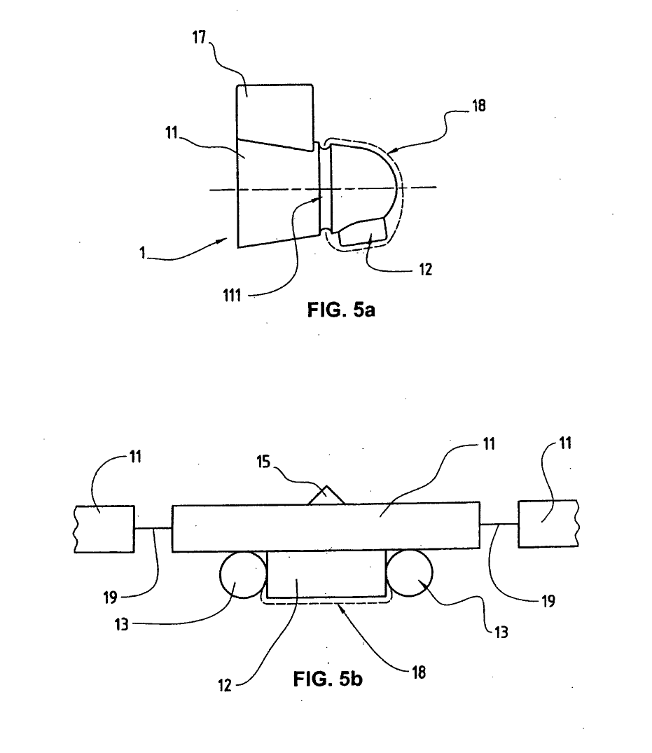

As is shown in FIGS. 1, 2 and 5b, the sensor 12 is encircled by an application ring 13 which, for example, is designed as a flexible rubber tube and serves as an application aid. As is shown in FIG. 5b, the application ring 13 can also be provided with a protective membrane 18 which covers the sensor 12. The protective membrane 18 is, for example, a thin and flexible latex, teflon, mylar or nylon membrane which does not influence the measurement. The application ring 13 provided with the protective membrane 18 serves as a fastening means for fastening the protective membrane 18 and can be pushed, preferably removably, over the sensor 12 so that it rests on the sensor 12 and the protective membrane 18 covers the sensor 12.

The protective membrane 18 can also be connected with a form fit to the measurement device 1 for covering the sensor 12. As is shown in FIG. 5, the support 11 designed as a thimble can, for example, be provided with a peripheral groove 111 which serves for fastening of a protective membrane 18 designed as an expandable protective cap.

Finally, the membrane 18 can be made at least partially of a self-adhesive material, so that, for the purpose of covering the sensor 12, it can be connected to the device 1 easily and removably.

In one embodiment variant, the sensor surface (with the sensitive region of the sensor 12) and/or the application ring 13 are of a concave design, for example like a contact lens. As a result of the surface tension of the lachrymal fluid, the sensor 12 is thus able to center itself upon application, by automatic suction. If a sufficiently elastic bearing of the sensor 12 is used, it is possible, after application, to decrease the application force in order to reduce the effect which finger movements have on the measurement.

In conclusion, it should be added that, in addition to the fastening means which have been described, the skilled person can provide the support 11 with other means for fastening to one or more fingers of the user. In particular, adjustable fastening means such as spreader rings, elastic bands or adhesive closures, for example velcro-type closures, permit fastening of the measurement device 1 to different fingers of different size and/or to several fingers.

1. Device for detecting measurement data of an eye, in particular a human eye, which device comprises a support and a sensor fixed to the support for detecting measurement data on the eye, the support being provided with fastening means for fastening the device to at least one finger of a user, wherein the sensor comprises a pressure sensor array.

2. Device according to claim 1, wherein the support is designed such that the sensor is positionable in the region of the fingerpad of a finger, the sensitive region of the sensor being remote from the fingerpad.

3. Device according to claim 2, wherein the sensor is fixed to the support in such a way that in the state of the support fastened to the finger a pressing force arising during an application of the sensor to the eye is transmittable to the finger.

4. Device according to claim 2, wherein the device comprises structural elements which are disposed behind the sensor and remote from the sensitive region of the sensor, and which are perceivable on the finger by the user in the state of the support fastened to the finger.

5. Device according to claim 2, wherein the device comprises an application ring which encircles the sensor and which abuts the eye during the detection of the measurement data.

6. Device according to claim 2, wherein, in the state of the support fastened to the finger, the length of the part of the support situated in the region of the fingerpad is limited to the length of the distal phalanx, situated there, of the finger.

7. Device according to claim 2, wherein the sensor is movably mounted on the support.

8. Device according to claim 2, wherein it comprises means for attaching a disposable protective membrane for covering the sensor.

9. Device according to claim 2, wherein it comprises an interface module fixed to the support for data communication, with contact or contactless, with an evaluation unit external to the device.

10. Device according to claim 2, wherein it comprises processing means, a data store, a display and/or an energy source, and in that the processing means, the data store, the display and/or the energy source are fixed to the support.

11. Device according to claim 2, wherein the support is designed as a bow comprising a curved area which rests on the finger tip in the state fastened to the finger.

12. Device according to claim 2, wherein the support is designed as a thimble.

13. Device according to claim 2, wherein the fastening means comprise a fastening clamp, an adhesive closure, an elastic band, a ring or a spreader ring.

14. Device according to claim 2, wherein the sensor further comprises a force sensor, a contact sensor, a distance sensor, a chemosensor, a surface sensor, a temperature sensor and/or a micro-optical emitter-receiver module.

15. Device according to claim 2, wherein the sensor comprises a light source as an optical application aid.

16. Device according to claim 2, wherein it comprises an electro-acoustical converter as an acoustical application aid.
